(19) United States
(12) Patent Application Publication
(10) Pub. No.: US 2009/0263487 A1
(43) Pub. Date: Oct. 22, 2009

(54) Bubble drink provided by bubbling engineering progress

(76) Inventor: Tae-Gook Kwon, Seoul (KR)

Correspondence Address: THE NATH LAW GROUP, 112 South West Street, Alexandria, VA, 22314, US

(21) Appl. No.: 12/457,180

(22) Filed: Jun. 3, 2009

Related U.S. Application Data
(63) Continuation of application No. 12/199,600, filed on Aug. 27, 2008, now abandoned.

(30) Foreign Application Priority Data
Feb. 28, 2007 (KR) 10-2006-0019468
Feb. 28, 2007 (KR) PCT/KR2007/001040

(51) Int. Cl. A61K 9/14; A23L 2/38

(52) U.S. Cl. 424/489; 426/569; 426/452

(57) ABSTRACT

A bubble drink as a health food, which is produced by imparting a catalytic function for gas escaping to an adsorptive fine food powder to allow the fine food powder to be dispersed in a colloidal solution reverse vessel, and reacting the dispersion with a gas-saturated solution to generate foam (a bubbling engineering process), whereby the food is converted into foam having a three-state composite structure of gas, liquid and solid states suitable to be ingested within a digestive system.

### BACKGROUND OF THE INVENTION

1. Field of the Invention

The present invention relates to ‘Bubble Drink’ or ‘Bubble Introducing Technology and the Related Goods,’ which best describes the unconventional conception of food ingestion that is defined and described hereinafter.

The invention is intentionally designed to create a methodology to convert the state of ‘food’ or ‘meal and drink’ into bubble formation for the purpose of effectively ingesting a considerable amount of beneficial gas materials and thereby promoting the organoleptic effectiveness on an organism of the ingesta including nutritious and beneficial facts taken into a living body.

The term of ‘Bubble Drink’ is coined, defined and employed for the sake of simplicity in explanation of the core concept of this inventive engineering design, which hereinafter is used in wider application than that of the literal and conventional synthesis of each word since it includes definitions of ideas like ingestion pattern of bubbled material, bubbling phenomenon, bubbling process, and/or the produced goods related to realize such bubbled ingesta.

Bubble Drink products by this invention can be served by impromptu (improvised) cuisine of simply mixing two major functional materials, i.e. a gas-saturated drink and a food concentrate in colloidal dispersion state and hence spontaneously forming a fluent complex aggregation of gas bubbles, and then are ingested by means of drinking in the form of synthetic construction as named ‘Bubble Drink’ which enables ingesta not only to help the living subject to keep and/or improve health but also to attain various functional effects when carefully designed and controlled. Those beneficial results are achieved by the property of bubble drink maximizing the introduction of gas within the digestive system with a soft feeling of gulp, thereby increasing the functional efficiency of the ingested gas materials.

2. Description of the Background

The current atmosphere of the earth consisting of oxygen, nitrogen, carbon dioxide, hydrogen, argon, etc. has been changing rapidly from its previous equilibrium state according to temperatures and partial pressures into a new one, one particularly with the increase of carbon dioxide ratio due to the excessive burning of fossil fuel resources over the past few centuries. This phenomenon is may have also contributed to the increase in various kinds of adult disease such as hyperpiesia, diabetes, obesity, cancers, and poor immune reaction syndromes observed in children and, in part, in plants and animals.

Considering the matter of keeping health in such hardships by the global transition atmosphere, it is necessary to develop such unconventional food commodity utilizing bubble structures in order to control the organoleptic partial pressures of ingested gas materials. Especially, carbon dioxide is now spotted as a major contribution to the indications of global warming and is regarded as a nuisance industrial waste, and the amount of carbon dioxide in the atmosphere is only increasing.

When we want to solve these problems, i.e. deterioration of bio functions due to the increase of atmospheric partial pressure of carbon dioxide and hence global warming, and to promptly induce a stable equilibrium of the atmosphere, it would be desirable to reduce this problem by building up a quick circulation system enough to feed back the carbon waste as value added industrial resources for the carbonated bubble drink food markets. In other words, we might imagine the idea of ingesting carbon dioxide highly valued in bio-function which is captured into the food bubbles, and then we could solve such problems altogether and easily.

On the other hand, there are a few examples of foamy drink present on the market such as carbonated drinks (soda pops) which are in form of aqueous solution manufactured by dissolving gas at low temperature and high pressure and draft beers which are in form of foamy colloid by the pressure generated during fermentation. When such conventional drinks are exposed to the ambient pressure and temperature, most of the dissolved gas is instantly isolated from the solution of the drinks and transformed into a foam or a draft of bubbles, whose solution we estimate is to be mostly ingested and whose ingesting system is for neither the gas nor the foam nor the draft of bubbles.

Even though part of the ingredients of a solution may be transformed into foam on the process of manufacturing the conventional drinks or according to their ingestion conditions, the major purpose for their ingestion stands far apart from drinking foam, and so, they are not to be included in the definition of ‘bubble drink’ in a strict sense of the present invention.

In the meanwhile, a few researches regarding foamy food has been conducted, for example, Food Emulsions and Foams (Based on the proceedings of an International Symposium organized by the Food Chemistry Group of The Royal Society of Chemistry at Leeds from 24th-26 Mar. 1986; Edited by Eric Dickinson, Procter Department of Food Science University of Leeds, England), which reports on whipped cream.

Also, the characteristics of particulate, amphiphilic and biological surfaces are mentioned in a recent study report of Colloid-Polymer Interactions (Edited by Paul L. Dubin and Penger Tong, Developed from a symposium sponsored by the Divisions of Polymer Chemistry, Inc., and of Colloid and Surface Chemistry at the 203rd National Meeting of the American Chemical Society, San Francisco, Calif., Apr. 5-10, 1992/ACS Symposium Series 532). The important fundamental principles employed for those researches have been already rooted in the studies of colloids and surface chemistry in the long time history, and have supported the main theories contributing to the opening of the field of science named modern polymer chemistry.

In connection with the definition of colloids described in “colloids and surfactants” (revised edition, Kook Yun-Hwan, a Doctor of Engineering, et al. Mar. 5, 1997), the characteristics of colloidal particles are described as follows: “Accordingly, fine particles in a colloidal solution are present in a dispersion state, irrespective of the properties of constituent materials, but critically defined only by their size limits which approximately ranges from 1 nm (1 nm=10⁻⁷ cm) to 1 μm (1 μm=10⁻⁴ cm), and the solution is referred to as “colloidal dispersion system”. In connection with the size of the dispersed particles, the reference explains the difference between the terms ‘suspension’, ‘colloidal suspension’ and ‘solution’ in “Table 1-4. the sizes of dispersed particles.”

From the viewpoint of the size criteria of dispersed particles, a colloidal solution constituting bubble drink can be defined as ‘complex colloid’ in which the three types coexist. By the definition of colloid, accordingly, the physicochemical properties of bubble drink are dependent on the sizes of the constituent materials and irrespective of those properties and thereby, the term ‘complex colloid’ naturally secures a wide variety of choice in identifying bubble drink material constituents, thus excluding the need for additional longwinded explanation thereof.

However, sugar is naturally mixed with milk, but aggregation occurs between a parched cereal powder and milk. Further, after a certain period of time when the sugar particles are completely dissolved and the parched cereal powder is aggregated and/or precipitated only to absorb moisture and swell, the electrostatic force in the contact surfaces of the particles must be lost and all the fine pores are completely filled, thus leaving no room for reserving or potentiating the designed time-differentiated reaction programming on the material.

### SUMMARY OF THE INVENTION

When the materials are processed in accordance with the procedures of bubbling engineering process, proposed in the present invention, the above-mentioned problems will be solved and thus a food concentrate is able to be prepared in the state of colloidal solution, which is a sol state dispersed with a suspension of quasi-polymer material where a reaction programming for bubble drink can be reserved or potentiated.

It is one object of the present invention to provide a new concept of ingestion of food to maintain or improve the health of lives using bubble drink wherein the bubble drink is produced by converting materials to be ingested present in a composite state of solid, liquid and gas states into a foam structure.

It is another object of the present invention to provide bubble drink produced by bubbling engineering using food and drink wherein ingredients beneficial to and suitable for human health are selected and the contents of the ingredients are controlled to provide a custom-made or custom-ordered product depending on the demand and taste of consumers.

More specifically, the foam of the bubble drink according to the present invention is an aggregate of fine air bubbles consisting of cells with a diameter of 2 mm or less. Most of the cells are maintained for at least 2 to 5 minutes without any aggregation or rupture of the cells. Since 80 to 90% of the bubbles are preserved during decanting of a container containing the bubbles exposing to the ambient temperature and pressure, bubble drink provides a feeling of refreshment and achieves a soft feeling of the foam in the throat together with desired taste and flavor when drinking it using a cup or glass in the presence of mind.

### BRIEF DESCRIPTION OF THE DRAWINGS

FIG. 1 is a conceptual view showing a system for producing a mixed powder which is used to prepare a colloid for a food concentrate of a bubble drink.

### DETAILED DESCRIPTION OF THE PREFERRED EMBODIMENTS

A conceptual comparison between bubble drink and conventional foamy drink is presented as follows.

As for basic materials in the present invention, generally, and in the present invention, parched cereal powder constitutes a suspension type (10,000 Å or more), food such as milk, partially a colloidal suspension type (10-10,000 Å), and sugar, a solution type (1-10 Å) of dispersed particles.

In the course of the production of conventional drinks, foaming is present as by-product or for visual effect or taste, but in the final stage, their drink for ingestion is still in liquid state, and there are no optional choices of the environment or conditions of new ingredients being added to change the state and texture of the material to be ingested under which a custom-made or custom-ordered product can be provided according to the demand and taste of consumers, from which the bubble drink of the present invention is distinguished.

For example, the foam of refreshing drinks can make a visual fresh effect and a pungent feeling, that of beer is a by-product of fermentation process, and ice cream is a simple artificial product with fine foam structure just for the purpose of soft taste.

In contrast, the invention concept of bubble drink discloses that the primary processing food material into the powder catalyst form purposes foaming, the secondary processing the primary powder into the complex colloidal suspension prepares for food concentration, and reacting the concentrate with a saturated aqueous solution containing gas so as to convert all reactants into a bubbled structure, thus allowing the ingestion of gas in the form of bubbles which is the concept of ‘bubble drink.’

In other words, bubble drink is a kind of instant food produced by converting materials to be ingested into a blast of bubbled structure in 3 state complexity of solid, liquid and gas. The invention will produce less occurrence of any cohesive trouble in each mixing step and easily contain considerable amounts of functional ingredients and accordingly, provide excellent taste and stability without causing any adverse side effect for drinking food containing functional ingredients in the form of bubbles.

The design of bubble drink was invented considering the common pattern of ingestion of all kinds of food and drink, and, so long as the basic requirements described herein are met, any food or drink undergoing a change in composition can be ingested in the form of bubble drink as an instant food which is produced by converting ingesta into a blast of bubbled structure in 3 state complexity of solid, liquid and gas even under the various influences of the actual life environment. An invention involving phase changes and exhibiting potent thermodynamic and quantum mechanical properties as stated in the present invention will never produce products having completely identical fingerprints. Furthermore, food ingestion environmental conditions cannot be manipulated just like those in laboratories and so potential instability and change cannot be avoidable. Under such environmental systems, an invention associated with a method for ingesting a physico chemically stable food or drink must ensure a consistency in the practice of the invention even under various and comprehensive daily life environments. With reference to technical and experimental data associated with the basic principles of the present invention and embodiments of the present invention, the technical spirits will be described below from the standpoint of the features and purposes of the present invention.

A method for ingesting food and drink in the form of bubble drink provided in the present invention is expected to originate a new culture of food ingestion. The term ‘bubble’ as used herein means an aggregation of different foam cells. The viscosity of colloid solution controls the 3 successive steps of capture, growth and maintenance, that membranes of constituent materials surround gas pores and then the foam cells are grown and maintained.

The present invention can employ the principle of dispersion catalyst's playing in aqueous solution of colloid in order to control the foam cells. In the present invention, the following conditions are set to obtain the desired status of bubble drink.

Firstly, gas and liquid (e.g., drink) and a solid (e.g., food powder) are converted into a blast of bubbles in the final step just before being ingested, with the food concentrate as a catalyst applied by a fall of gas-saturated drink to blast bubbles.

Secondly, a gas-saturated drink, in which a gas material, such as carbon dioxide (CO2) or oxygen (O2) in order to be applied to humans is saturated, is prepared for the purpose of producing bubble drink. (This may be replaced by a conventional gas-saturated product).

After the above requirements are met, Grain powder is made into glycoprotein colloidal gel composite. Thereafter, the composite colloidal gel state is converted into a food concentrate in the state of sol (e.g., a hydrosol or organosol). Accordingly, a catalyst function for gas escaping can be preserved in the food concentrate.

More detailed explanation of the catalytic function for gas escaping is as follows. Since the saccharine crystalloid powder is adsorbed to the grain powder, the surface adsorption potential of the grain powder is kept in the colloidal aqueous solution until before a final reaction. In order to impart a foaming function and/or a foam stabilizing function, the materials should be appropriately processed and mixed in the right order to store the reaction steps in the composition of raw materials such as colloidal structures, and then the reactions are sequentially carried out using a time difference, so that a reaction program can be restored or potentiated on the material.

Foam capturing is the function that can be realized to control the duration of bubbles and the size of foam cells according to the amount of milk input when manufacturing sol colloid.

In order to effectively accomplish the technical spirit of the present invention, it is important to pretreat raw materials of the bubble drink according to the present invention. The raw materials are prepared in accordance with the following procedure. A parched cereal powder as a basic raw material of the bubble drink according to the present invention is prepared by a well-known method.

Grains, which are used to produce the bubble drink of the present invention, are processed into fine powders. The grains are processed by primary pulverization, and finely pulverized using a pin mill, a jet mill, an impact mill, a roll mill and/or a colloid mill at a cut-off wheel speed (COWS) of 500 to 15,000 rpm to prepare a grain powder having size of 1 to 100 μm. Particles having a size of 100 μm or more, that cause formation of a precipitate or lump, deteriorate dispersibility or cause an unpleasant feeling of eating, are removed from the grain powder by air stream distribution.

A saccharine crystalloid powder or granule having a size of 10 μm or less as adispersion auxiliary may be prepared in the same manner as in the preparation of the grain powder. More advantageously, the dispersion auxiliary is prepared from a supersaturated solution.

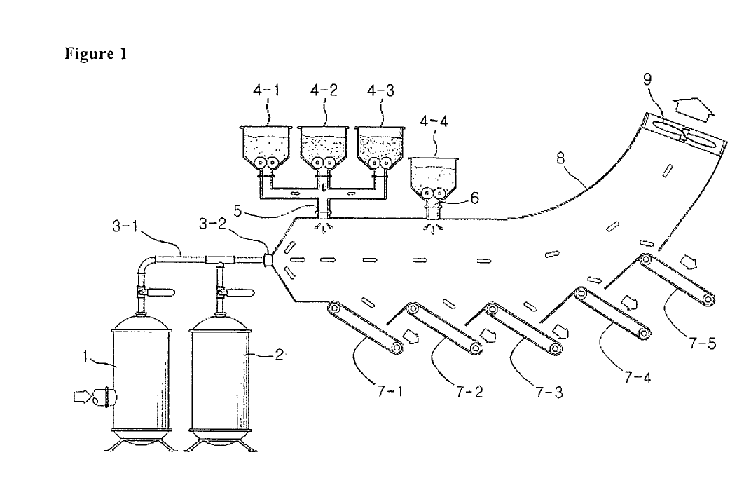

FIG. 1 is a conceptual view showing a system for producing a mixed powder of the grain powder and the dispersion auxiliary. In the system, dehumidification blast, wind tunnel mixing, adsorption and air stream distribution are performed. First, high-pressure low-temperature liquid carbon dioxide is sprayed to mix and adsorb the grain powder, the saccharine crystalloid powder, a dry ice powder and powders of a variety of additives. The mixture is distributed by air stream on the basis of size (i.e. >100 μm, 100 μm-10 μm, 10 μm-1 μm, and 1 μm-1 nm), escaped, collected, and packaged in a frozen state under vacuum. Specifically, a liquid carbon dioxide spray tank 1 and a tank 2 containing a supersaturated aqueous solution of a fruit juice, sugar juice, monosaccharide or oligosaccharide crystalloid are connected to a spray tube 3-1. A spray nozzle 3-2, which is installed at a front end of a closed wind tunnel 8, is connected to the spray tube 3-1.

A controller is provided on one side of the wind tunnel 8 to control the speed of air stream. A plurality of hoppers for feeding necessary materials to the air tunnel are provided with respective roll mills. Grains 4-1, a parched cereal powder 4-2 and additives 4-3 are fed to the air tunnel through the respective hoppers. The powders fed from the respective hoppers are collected and supplied to the wind tunnel 8. In an alternative embodiment, the grains are primarily pulverized, mixed with a coarse saccharine crystalloid powder, and secondarily pulverized to obtain a powder having a size of 10 μm to 100 μm.

Another controller is provided on one side of the wind tunnel 8 to control the speed of air stream. A hopper for feeding a crystalloid 4-4 to the air tunnel is provided with a roll mill. The crystalloid 4-4 is fed to the air tunnel through the hopper.

Air stream is used to distribute the powders. To separate and collect the distributed powders depending on particle size and weight; holes are formed under the wind tunnel and conveyer belts 7-1, 7-2, 7-3, 7-4 and 7-5 are installed at spaced intervals on the inclined surface of the wind tunnel. The number of the conveyer belts is varied according to the intended purpose of the distribution. Preferably, the conveyers are directly connected to an in-line system, where the powder materials collected through the respective conveyers can be closely packaged in a frozen state within a short time.

A propeller 9, whose speed and direction is controllable, is installed at the opposite end of the wind tunnel to increase or reduce the discharge pressure of the wind tunnel. A unit for capturing fractions on a nanometer scale discharged through the propeller together with the air stream may be installed.

In the course of adsorption using the wind tunnel, high-pressure low-temperature liquid carbon dioxide is sprayed to improve the performance of the wind tunnel so that the temperature, pressure, humidity, air stream distribution, particle size, and constituent ratio and binding state of the powders can be controlled. At this time, the carbon dioxide present in the state of a dry ice powder is mixed with the powders to improve the preservation of the food, thereby achieving powder processing in a frozen state under ambient pressure that ensures high transport stability.

A saccharine crystalloid powder having a size of 10 μm or less may be further added to the fine food powder to prepare the bubble drink.

The technical spirit of the present invention will be explained below in more detail.

In order to produce the products of this concept, a gas-saturated aqueous solution is used as a gas source and the reactions of a dry food powder as a catalytic agent for gas escaping are controlled through the design of material processing used: For example, a saccharine crystalloid powder is selected as a material for preserving an adsorptivity on the surface of fine powder of roasted grains or grain powder to keep the ability to explosively blast the fine powder of roasted grains into a blast of bubble colloid.

Further, a program is applied to reserve or potentiate reaction energy (i.e. foaming procedures) of the respective steps on the structure of reaction materials. For example, the program is composed of the following materials: [(parched cereal powder+granular powder containing saccharide ingredients and/or sugar powder+powder additives)+drops of milk+milk+liquid type additives]+a gas-saturated drink.

Further, a fine powder of a dry food is used as a reaction catalyst and a saccharine crystalloid powder is used as a dispersion medium of a composite colloid. A dispersion and diffusion derivative is used to crystallize the saccharine crystalloid powder into a hydrate (an amorphous crystal).

This hydrate crystallization technique is a technique wherein, for example, drops of milk are dripped and rolled on an inclined surface of a reserve vessel of the grain powder (the parched cereal powder+the saccharine crystalloid powder+the powder additive) to infiltrate the milk into the grain powder and then the reserve vessel is formulated into a pill to be a gel state.

Milk is added to the pill in a gel state to convert the gel into a sol, and as a result, a food concentrate (a sol colloid reserve vessel) in the state of a quasi-polymer solution is prepared. The food concentrate instantaneously captures an evolved gas (a bubbling engineering process). The bubbling engineering process is suitable for the generation of foam for a bubble drink.

The technical spirit of the bubbling engineering process employed in the present invention has been demonstrated through a variety of experiments. It has been found that salt, fermented foods (e.g., soybean paste, soy sauce, powder of soup prepared with fermented soybeans, and yoghurt), and edible food additives beneficial to human health (e.g., charcoal powder, pollen, honey, tobacco (ash) powder, vegetable enzyme materials, parched food materials, uncooked food materials, dietary fibers, starch, dry ice, coffee, ginseng steamed red, green tea, herbs, healthy foods, healthy food supplements, cold medicines, medicines prescribed and provided by doctors and pharmacists according to the kind of diseases, herbal medicines in the form of granules, extracts, medicines in draught, pills and powders, etc.) can be processed and ingested by the bubbling engineering process.

It has also found that when colloidal additives, for example, additives (e.g., flavors, various gases and liquid carbon dioxide) in the first step and a powder additive in the third step, were sufficiently mixed in accordance with the programmed procedures, the whole mixture acted as catalysts for colloidal explosion (rapid foaming phenomenon), and at the same time, a gas could be instantaneously captured in foam cells by the bubbling engineering process.

By appropriately mixing a dispersoid of the sol colloidal solution, the dispersion medium and the additives, foam in a desired form can be obtained within almost all food compositions that are used in dietary life of modem people. The present inventor has found that foam having an expanded diameter of 1 to 2 mm could be obtained within 1 to 3 seconds depending on the characteristics of the respective ingredients, the expanded foam could be maintained for about 3 to about 5 minutes, above 95% of the foam could be drink at one time, and no belching caused after drinking. The present invention has been accomplished based on these findings.

Further, it was found that when a parched cereal powder, which is a Korean traditional food material, a granular powder containing a saccharide ingredient and/or a mixture of a sugar powder and milk were mixed in a volume ratio of 1:1:2 (the parched cereal powder/the saccharide/the milk), an effective major colloid could be obtained. Further, it was found that various factors (e.g., temperature, functionality, convenience and continuity) of the foam could be controlled in accordance with the mixing and preparation methods of the powders depending on the taste of foods and the need of consumers.

The powders can be mixed by simple mixing methods (e.g., roll milling) by pulverization, mixing methods by centrifugation, mixing methods using a colloid mill and/or a wind tunnel, mixing methods by air stream distribution, and other mixing methods. The procedure may be varied and complemented. For example, dry ice and a charcoal powder are mixed with a fine powder of roasted grains and the mixture is mixed with a saccharine crystalloid powder to allow colloidal particles to have an appropriate size and a fine and uniform state.

Particularly, when the food powder as a foam-forming substance is processed or reacted, the ingredients of the food powder can be varied depending on the demand of consumers. As a result, various edible tea products, such as coffee, and powders of herbal medicines may be added depending on the propensity and demand of consumers to provide a custom-made or custom-ordered product. The production procedure can be manipulated in a simple manner so that various characteristics of functional drinks can be readily achieved.

Further, suitable functional ingredients may be added to enhance the role and taste of drinks for particular purposes. Examples of such drinks include therapeutic drinks, healthy drinks and other functional drinks, such as diet drinks and aromatic drinks. Additional techniques, such as fermentation, may be employed to prepare applied foods, such as functional fermented bubble drinks (bubble yoghurt), and realize various food cultures. Accordingly, the present invention is a useful invention that can open new value-added industries and can create a better value of life.

As mentioned above, the bubbling engineering process is a technique wherein the hydrophilic grain powder is dispersed in the state of a colloid to form fine foam. More specifically, the hydrophilic grain powder is doubly dispersed in a colloidal solution in a protein emulsion state to prepare a food concentrate in a composite colloidal solution of emulsion-suspension, and then the composite colloidal solution is instantaneously reacted with a gas-saturated aqueous solution, in which a gas, such as CO₂ or O₂, harmless to humans is dispersed and/or saturated in gas, liquid and/or solid states, to convert the entire ingredients to foam having a diameter of 2 mm or less. This technique is carried out to render the bubble draft fluent enough to drink and to retard the collapse of the foam cells even during transport, thus controlling the structure of bubbles to be without feeling awkward inside the digestive organ.

The Bubbling Engineering Process is divided into the following steps;

1st step: Preparation of gas saturated aqueous solution as for gas saturated drink.

2nd step: Steaming and drying or slightly parching natural grains to obtain a parched cereal powder, pulverizing the parched cereal powder into a fine powder of roasted grains for bubble drink while maintaining the humidity at a level lower than 5%, and dividing the fine powder of roasted grains into powders on the basis of size (>100 μm, 100 μm-10 μm, 10 μm-1 μm, and 1 μm-1 nm) for the purpose of its acting gas escaping agent against gas saturated drink.

3rd step: Among the Crystalloid materials, such as replacement, diffusion and dispersion derivative agents like monosaccharide, oligosaccharide and/or fruit juice granule are selected and processed into fine powder of size of 10 μm or less in diameter and then mixed with the powders prepared in the second step in a wind tunnel, adsorbed and distributed to obtain a powder having a regular particle size of 10 μm or less. In this step, in order to add functionality of food to the product, certain functional additives also go under the same process.

4th step: Preparation of Emulsion-suspension type of colloid solution role-playing as a colloid reserve vessel on which food concentrate may be made so that dispersion may reserve and foam stabilizing function on the basis of colloid suspension.

5th step: props of milk are dripped and rolled on an inclined surface of a reserve vessel of the grain powder (the parched cereal powder+the saccharine crystalloid powder+the powder additive) to infiltrate the milk into the grain powder and then the reserve vessel is formulated into a pill to be a gel state for the purpose of rapid diffusing of powder materials during the bubble blast occurs.

6th step: Pulverizing, rotating or swirling the food concentrate to convert the gel into a sol with additional supplying of colloid reserve vessel (i.e. milk) as for a dilute solution in order to function the 3 steps of viscosity effects as in protein foaming, saccharine crystalloid diffusion and starch adhesiveness. In this step, functional additives can be enforced.

7th step: With a container of the ambient temperature and pressure big enough to accommodate all the expanding blast of bubbling as a result of pouring of gas saturated drink over the food concentrate laid on the bottom, henceforth, the kinetic energy plays the role of triggering for the bubble blasting resulting from free-fall.

8th step: Tilting the container to make the draft of bubble flow into the right procedure.

The key technical spirit associated with the production of bubble drink according to the present invention includes the following techniques:

i) Natural grains are steamed and dried or slightly parched, pulverized to prepare a fine powder of the natural grains (i.e. a parched cereal powder). The parched cereal powder is pulverized into a fine powder of roasted grains for bubble drink while maintaining the humidity at a level lower than 5%. The fine powder of roasted grains is divided into powders on the basis of size (i.e. >100 μm, 100 μm-10 μm, 10 μm-1 μm, and 1 μm-1 nm);

ii) Moisture trapping effects of a saccharine crystalloid powder limiting the contact binding of a colloid reserve vessel with moisture are utilized to preserve a moisture adsorption potential of the grain powder when a powder dispersoid as a catalyst for gas escaping is stirred in the colloid reserve vessel (primary dispersion) before reaction with a gas-saturated drink as a target reactant of a corresponding dispersion medium (secondary dispersion);

iii) The saccharine crystalloid powder and the dry grain powder are blown in a wind tunnel to adsorb the saccharine crystalloid powder to the dry grain powder utilizing a surface adsorptive force and an electrostatic force. As a result, infiltration of the mixture into an aqueous solution, replacement with gas pores and viscosity of the membranes of air bubbles are enhanced;

iv) During the preparation of the crystalloid powder, the structure of the colloidal dispersion medium is designed in a composite manner such that the respective mixing reactions, such as replacement, diffusion and dispersion, are controlled according to the purpose of additives. Specifically, the saccharine crystalloid powder is divided into powders on the basis of size (100 μm-1 nm) and hydrated. The hydrate is converted into a gel state in a colloid reserve vessel in the state of emulsion-suspension, and diluted to prepare a sol. As a result, the structure of the gel colloid is aggregated and aged with the passage of time;

v) Immediately after the gas is evolved in the form of fine structures, a viscous foamed solution is prepared. The grain powder and the saccharine hydrate crystal powder are infiltrated into the protein colloidal emulsion to be attached to the protein structure, thereby inducing a quasi-polymer state. Then, when exposed to a gas carrier, the quasi-polymer state and the saccharine crystalloid powder exchange the gas ingredient to rapidly induce formation of fine air bubble cells in the protein reserve vessel;

vi) Free fall and vortex turbulence are employed as aeration triggers for colloidal explosive reactions. Accordingly, the height and amount of the free fall are controlled to adjust an increase in the entropy of the bubble colloid, and at the same time, a gravity-free state is imparted to the gas-saturated drink to achieve uniform and fine foaming during the free fall;

vii) An excess of gas are continuously captured within the foamed air bubbles without any fusion of the foamed air bubble cells to induce growth of the air bubbles into foam having a suitable size; and

viii) Three steps for enhancing the viscosity of the foam are carried out such that the foam can be appropriately maintained in a stable state.

Based on the foregoing techniques, the following suitable materials are selected, processed and mixed to provide the bubble drink of the present invention.

The gas escaping must be vigorously conducted at a high rate in order to form fine air bubbles, and the fine air bubbles must be directly captured and maintained in drops of a liquid before growth of the air bubbles. The present invention employs a principle wherein a gas dissolved in an aqueous solution is rapidly foamed and violently discharged when a highly adsorptive fine powder is used as a catalyst in the aqueous solution. To this end, a highly adsorptive parched cereal powder as a natural food is dried to have a moisture content lower than 5%, and is mixed with a finely divided saccharine crystalloid powder having a size of 10 μm or less in a volume ratio of 1:1 to adsorb the saccharine crystalloid powder to the parched cereal powder. The mixture thus obtained is processed to prepare a catalyst dispersion material for gas escaping.

The grain material becomes highly viscous in a colloidal state. At this time, the amount of glutinous rice flour added is varied depending on that of a material of the gas-saturated aqueous solution to increase the viscosity so that the grain material can capture the evolved gas for a long time. Through the mixing of a protein reserve vessel in the final step to prepare a concentrated colloid, the following functions are provided stepwise by the increased viscosity.

First step: The viscosity of milk, which is mixed with a hydrophilic saccharine crystal to prepare a colloid reserve vessel, is increased at the stage of formation of fine air bubbles. By this increased viscosity, membranes of the fine air bubbles are formed immediately when the air bubbles are formed. Then, membranes primarily blocking external air are formed to protect and maintain the foamed gas in a pure state.

Second step: Saccharine is very rapidly dissolved and diffused along the viscous membranes of the air bubbles, induces diffusion of the grain powder, evolves a large amount of gas, and imparts an elasticity so that the fine air bubbles can grow into foam having a suitable size.

Third step: When the fine powder of roasted grains, which constitutes a colloidal dispersion, is reacted with water for a sufficient time, it exerts an efficacy as a starch paste and maintains the viscosity of the already evolved fine air bubbles for a long time.

In a simple embodiment of the present invention, the food concentrate of the composite colloid in the state of emulsion-suspension, which is the final material as a catalyst for the formation of bubbles, can be easily prepared from natural food materials available in daily life. The foregoing is summarized below to assist in a further understanding of the present invention.

On the other hand, it is necessary for the catalyst powder particles to rapidly contact and react with the saturated aqueous solution, from which a gas is evolved. The catalyst powder particles are sprayed on or mixed with the aqueous solution, aggregation and isolation phenomena occur. As a result, it is impossible to expect effective catalytic functions of the catalyst powder particles and it is difficult to uniformly control the reaction time and the size of foam.

Further, when stirring is conducted using a physical force, inconvenience is caused and the state of the foam is adversely affected, resulting in deteriorated quality of the drink. Accordingly, the medium, where the catalyst acts, may be varied to program a reaction procedure in the characteristics of the reaction materials so that the reaction procedure can be controlled. That is, the composition of the materials can be designed such that the reactions can be automatically and sequentially conducted. In view of the foregoing, the catalytic reactions of the fine powder are conducted in a colloidal solution similar to an aqueous solution to achieve simple use and desired characteristics.

First, depending on the characteristics of the gas carrier (i.e. the gas-saturated drink), the size and the size distribution of the colloidal particles used as catalysts of the bubble drink are optimally varied to control the size of the air bubbles. When the colloidal particles are reacted with an aqueous solution, the colloidal structure is induced to a dispersed state of a composite multilayer structure so that dispersion and diffusion are further activated, and as a result, the adsorption potential is maintained.

To this end, milk can be used as a reserve vessel of the colloidal solution. Milk is a healthy food and is in the state of a protein colloidal emulsion. Milk can be used as a dispersion medium in the present invention. When milk is mixed with the fine powder, the milk is used is in an amount about two times greater than that of the fine powder so that the milk is sufficiently diffused into the colloidal state. Since a crystalline powder of a hydrophilic and highly soluble saccharine, such as sugar, is homogeneously mixed with the uniform and fine powder particles by stirring, the preservation of the food is improved, and the infiltration of moisture and the diffusion into a solvent during reaction with an aqueous solution are enhanced. Foaming is achieved by the following five-step mechanism:

First step: A hydrate ingredient of the saccharine crystalloid powder, which binds to moisture of a protein colloidal emulsion present in milk, is lost while it comes in contact with the gas carrier. At this time, a gas isolated from the gas carrier occupies the vacant spaces where the hydrate is lost, and as a result, air bubble cells are formed and swollen with the help of protein ingredients of the milk. The air bubbles at the initial stage act to form membranes primarily blocking external air to protect and induce growth of subsequently formed foam.

Second step: The food concentrate is reacted with the gas carrier while it is in uniform contact with the gas carrier to allow the hydrate of the saccharine crystal surrounding the grain powder to absorb moisture and to be rapidly diffused into the gas carrier;

Third step: While the saccharine is dissolved and diffused, it disperses the grain powder to evolve a gas from the gas carrier, thereby rapidly generating fine air bubbles;

Fourth step: The ingredients of the grain powder absorb water to further separate a gas from the gas carrier and increase the elasticity of the solution to grow the air bubbles; and

Fifth step: The grain powder, whose contact with moisture is limited in a colloidal state, absorbs a sufficiently large amount of water to exert an adhesiveness (which is enhanced by ingredients of glutinous rice flour) like a starch paste. As a result, the adhesiveness leads to an increase in viscosity, so that the durability of the foam films is enhanced and the foam is maintained until drinking.

By adding suitable materials during the preparation of the colloidal solution designed above and violently mixing with an aqueous solution, the taste, fragrance and functions of the final drink are controlled and enhanced in a very easy manner.

That is, the control of the foam-forming catalyst is more effective and simpler than that of the gas carrier. Particularly, foam functions to preserve a fragrance, e.g., xylitol, for a long time and to emit an aroma through the oral cavity for a long time after ingestion. Therefore, it is believed that the bubble drink is most effective in producing aromatic diet drinks.

Further, various tastes of people can be reflected according to the kind of a material added to colloidal particles as dispersion media and the reserve vessel material as a dispersoid in the state of an aqueous solution. Furthermore, it is very easy to mix the drink with at least one hygienic and physiological material selected from aromatic ingredients, healthy food ingredients and therapeutic ingredients (e.g., cold medicines, drugs for promoting blood circulation, internal medicines for treating hypertension, internal medicines for treating tinea pedis, etc) and to take the mixture.

The bubble drink of the present invention provides the following effects. The freshness of the bubble drink in the form of foam can be maintained for a long time. Conventional foamy drinks offer inconvenient stimuli and an awkward feeling, whereas the bubble drink of the present invention solves the problems of inconvenient stimuli and an awkward feeling, and at the same time, achieves a soft feeling in the throat, thereby providing a comfortable feeling and enabling effective ingestion of gas.

In addition, since the bubble drink of the present invention contains a calorie-free gas and a small amount of a food, it relieves thirst and regulates appetite. Therefore, the bubble drink of the present invention can be used as a diet food for preventing and treating diseases, such as obesity and diabetes. Furthermore, foam generated by the bubble drink of the present invention functions to preserve a fragrance for a long time and to emit an aroma through the oral cavity for a long time after ingestion. Therefore, the bubble drink of the present invention is effective in producing aromatic drinks.

A colloidal solution of the bubble drink is prepared by dispersing the mixture in a composite colloidal solution of emulsion-suspension to obtain a food concentrate in a composite colloidal state and instantaneously reacting the food concentrate with a solution saturated with CO₂ to convert the entire solution into foam having a diameter of 2 mm or less.

The gas-saturated solution of the bubble drink contains CO₂ in the state of a gas, a dry ice powder, a liquid or a composite thereof.

Preferably, a functional material is added during mixing of the parched cereal powder and the saccharine crystalloid powder. The kind of the functional material is dependent on the intended drinking purposes, functions and tastes.

Solid powders, liquids and gases may be added during mixing. Any natural material or processed material may be added without substantial limitation. A dry ice powder may also be added.

Specific examples of such additives include: powders of a variety of nutrients, including starch, proteins, fats and vitamins; powders of biologically active substances, including animal and vegetable sterols, amino acids, fatty acids and hormones; powders of minerals, including iron, manganese, sodium, magnesium, calcium, phosphorus, sulfur, germanium and trace elements, e.g., selenium; powders of inorganic materials, including tourmaline powder, loess, charcoal and tobacco ash; crystal powders of metals, such as gold, silver and platinum; rock powders; raw mineral powders; mixing-processed powders; fermentation-processed powders; fermented substance-mixed powders; and combinations thereof.

Since these biologically beneficial substances achieve an optimal balance provided by nature, they are in harmony in terms of biochemistry, molecular biology and/or biotechnology. Therefore, by utilizing an ergonomic data transmission system that imparts psychological communication effects, the substances are provided as good tasting materials for a bubbling engineering process.

According to the present invention, there is provided a method for producing a bubble drink that is provided as a custom-made or custom-ordered product to maintain or improve the health of lives and is ingested for various purposes, the method comprising the steps of:

steaming and drying or slightly parching natural grains, and pulverizing the dried or parched natural grains to prepare a fine powder of roasted grains having a humidity lower than 5% (first step);

pulverizing a crystalline powder or granular crystal of a monosaccharide or oligosaccharide selected from solid substances including sugar, fructose, glucose, lactose, starch sugar, oligosaccharide, dextrin, -starch, D-mannitol and xylitol to prepare a saccharine crystalloid powder having a size of 10 μm or less (second step);

adsorbing and distributing the powders prepared in the first and second steps in a wind tunnel to obtain a powder having a particle size of 10 μm or less, adding a functional material to the powder, and mixing the mixture with a colloidal solution in the state of a protein emulsion, such as milk, to prepare a food concentrate in a gel state (third step); and

pulverizing, rotating or swirling the food concentrate to convert the gel into a sol, and freely dropping a gas-saturated solution on the sol to generate foam (a bubbling engineering process) (fourth step).

In the third step, the functional material may be processed into a state of a fine powder or colloidal solution suitable for a bubbling engineering process before addition. In the third step, the gelling is carried out by dripping and rolling drops of the colloidal solution on an inclined surface of the powder to infiltrate the colloidal solution into the powder.

In the fourth step, the bubbling engineering process is carried out through the sub-steps of processing the raw materials into a powder having a particular particle size, dispersing the powder in the colloidal solution, and reacting a gas-saturated solution with the colloidal dispersion.

In the fourth step, the free fall is preferably performed in a gravity-free state. According to the present invention, there is provided a food concentrate of a bubble drink, the food concentrate comprising the following ingredients:

a fine powder of roasted grains having a humidity lower than 5% prepared by steaming and drying or slightly parching natural grains, and pulverizing the dried or parched natural grains;

a saccharine crystalloid powder having a size of 10 μm or less prepared by pulverizing a crystalline powder or granular crystal of a monosaccharide or oligosaccharide selected from sugar, fructose, lactose, starch sugar, oligosaccharide, dextrin, α-starch and D-mannitol; and

a food concentrate in a gel state prepared by mixing an additive with a colloidal solution in the state of a protein emulsion, such as milk, to impart desired functions to the bubble drink and gelling the mixture.

The bubble drink is a functional food provided for a variety of purposes, such as maintenance and improvement of the health of lives. By imparting a catalytic function for gas escaping to an adsorptive fine food powder to allow the fine food powder to be dispersed in a colloidal solution reverse vessel and reacting the dispersion with a gas-saturated solution to generate foam (a bubbling engineering process), the food is converted into foam having a three-state composite structure of gas, liquid and solid states so as to be ingested within the digestive system.

Embodiments of the present invention for achieving the technical spirit of the present invention can be provided in various different manners. The following examples are given to make the practice of the present invention easier. In the following examples, four basic ingredients, i.e. an aqueous solution of CO₂, milk, sugar, and a parched cereal powder (CPC, specially expressed as ‘fiporog’ in the case of a fine powder of roasted grains having a size of 10 μm or less prepared by the bubbling engineering process employed in the present invention) were selected and used. It was found through experiments that although various additives having different materials and compositions thereof were used for various purposes to produce bubble drinks, the bubble drinks showed similar effects without significant differences in terms of their physical properties.

This finding proves that the bubble drink of the present invention has stable and consistent physical properties, irrespective of the nature and mixing of the materials used. The following examples are not intended to limit the intrinsic principle and constitution of the present invention as disclosed in the accompanying claims.

In a simpler method, a flavored carbonated drink was mainly used as a gas carrier. The flavor carbonated drink can be prepared by any well-known method. Mineral water (CO² content: 1.112%) produced from Chojeong-ri, Chungcheongbuk-do, Korea, natural soda pop, and flavored carbonated drink products, including Coca-Cola Zero, Kin Cider, Fanta and Demisoda, were used in the following examples. All drink products were stored in a freezer at 5° c.

The volume of each of the carbonated drinks was measured in a cylindrical container having a diameter of 9 cm and a height of 9 cm at ambient pressure and room temperature. The height of each of the carbonated drinks was measured in a glass having a height of 12 cm and a diameter of 6 cm, which is routinely used at home. A colloidal solution (milk+powder of roasted grains+sugar) was added to the glass, and a gas carrier fell freely from a height of 30 cm within 5 seconds to induce turbulence. As a result, a bubble colloid was obtained. The maximum volume of the bubble colloid was expressed in Vmax.

The milk can be prepared by an ordinary technique. In the following examples, E+ Supgol Milk (provided by FamilyMart Co., Korea), Pasteur Fresh Milk (produced by Pasteur Milk Co., Korea) and Pasteur Organic Milk (produced by Pasteur Milk Co., Korea) were used.

A mixture of honey, egg, mayonnaise, butter, soybean soup, sesame oil and yoghurt, both of which are in a colloidal state, as edible additives was used. The addition of butter and sesame oil caused a reduction in foaming function.

The parched cereal powder, a parched food powder and a powder of vegetable enzymes were readily prepared by well-known techniques. In the following examples, three powders of different types were used.

Parched cereal powder A (fiporog A): Unhulled barley (37.5%), brown rice (25%), brown glutinous rice (18.7%), black soybean (16.3%), and others (chestnut, sea tangle, etc). Parched cereal powder B (fiporog B): Barley (27%), brown rice (25%), corn (25%), brown glutinous rice (10%), black soybean (10%), and others (potato, sweet potato, sea tangle, etc) Parched cereal powder C (fiporog C): A parched cereal powder for parched food, which was prepared by processing a mixture of a parched cereal powder and dry parched food materials wherein the parched cereal powder consists of brown glutinous rice (13%), barley (13%), unhulled barley (15.%), brown rice (13%), black soybean (13%), white soybean (4.4%), unshelled grains of adlay (4.4%), African millet (4.4%) and corn (4.4%) and wherein the dry parched food materials consist of sesame (2.2%), black sesame (2.2%), wild sesame (2.2%), sweet potato (0.88%), potato (0.88%), sea tangle (0.44%), anchovy (0.44%), brown seaweed (0.44%), chestnut (0.88%), mushroom (0.44%), spinach (0.44%), cabbage (0.44%), mugwort (0.44%), onion (0.44%), banana (0.44%), an embryo bud of brown rice (0.88%), pumpkin (0.44%), carrot (0.44%) and apple (0.44%).

For better taste, nutrition and function, edible additives were mixed, for example, starch flour, starch, york flour, parched wild sesame flour, coffee extract powder, salt powder, green tea flour, powder of ginseng steamed red extract, pepper flour, powder of soup prepared with fermented soybeans, dry ice powder, powder of various vegetable enzymes, powder of herbs and pollen.

As the sugar, white sugar having a diameter of 1 mm or less was mainly used. The sugar was mixed with the parched cereal powder, and then the mixture was pulverized into a fine powder (fiporog A100) having a size of 100 μm or less and a fine powder (fiporog A10) having a size of 10 μm or less. Although mannitol or xylitol was further added or used instead of the sugar, similar results were obtained.

To measure the degree of separation of the structures, the ratios of a solution state to a foamy state separated from a 100% foamy state with time (0.5 min., 1 min., 5 min., and 10 min.) were expressed as R0.5, R1, R5 and R10, respectively. One method selected from the volume and height measurement methods was employed to measure the degree of separation. Specifically, the ratios were expressed as values of Vt (total): Vl (liquid): Vb (bubble) in ml or values of Ht (total): Hl (liquid): Hb (bubble) in cm. In particular examples (Fanta/cake production and purification functions of contaminants), the degree of clearness of the separated solution state with the passage of time was measured, relative to the degree of clearness of background letters (20 points). The results were evaluated based on the following criteria: Good (easily discernable), Average (discernable with difficulty), Poor (indiscernible).

### EXAMPLES

### Example 1

When 250 ml of a gas-containing aqueous solution alone fell freely, the following measurement results were obtained: Vmax=500 ml, R0.5 (V)=250:250:0, R1 (V)=250:250:0, R5 (V)=250:250:0, R10 (V)=250:250:0.

### Example 2

When 250 ml of a gas-containing aqueous solution (Mineral water produced from Chojeong-ri, Chungcheongbuk-do, Korea) fell freely down 100 ml of Pasteur Fresh Milk (produced by Pasteur Milk Co., Korea), the following measurement results were obtained: Vmax=800 ml, R0.5 (V)=750: 200:550, R1 (V)=600:270:330, R5 (V)=370:340:30, R10 (V)=350:350:0. At this time, no creamy ingredient of the milk was extracted.

### Example 3

When 125 ml of a gas-containing aqueous solution (natural soda pop) fell freely down 50 ml of E+ Supgol Milk (provided by FamilyMart Co., Korea), the following measurement results were obtained: Vmax=400 ml, R0.5 (V)=400:80:320, R1 (V)=350:120:230, R5 (V)=290:160:130, R10 (V)=230:170:60. About 30 minutes after the free fall, a creamy ingredient of milk was extracted.

### Example 4

5 g of fiporog A-100 was homogeneously mixed with 5 g of sugar to obtain a powder. The powder was added to 50 ml of E+ Supgol Milk (provided by FamilyMart Co., Korea) to prepare a composite colloidal solution. When 125 ml of a gas-containing aqueous solution (natural soda pop) fell freely down the composite colloidal solution, the following measurement results were obtained: Vmax=400 ml, R0.5 (V)=400:80:320, R1 (V)=380:120:260, R5 (V)=350:150:200, R10 (V)=330:160:170. About 30 minutes after the free fall, a solid structure in the form of a foam crist was obtained.

### Example 5

5 g of fiporog A-10 was homogeneously mixed with 5 g of sugar to obtain a powder. The powder was added to 50 ml of E+ Supgol Milk (provided by FamilyMart Co., Korea) to prepare a composite colloidal solution. When 125 ml of a gas-containing aqueous solution (natural soda pop) fell freely down the composite colloidal solution, the following measurement results were obtained: Vmax=400 ml, R0.5 (V)=400:100:300, R1 (V)=380:120:260, R5 (V)=350:150:200, R10 (V)=330:160:170. About 30 minutes after the free fall, a solid structure in the form of a foam crust was obtained.

### Example 6

5 g of fiporog B-100 was homogeneously mixed with 5 g of sugar to obtain a powder. The powder was added to 30 ml of E+ Supgol Milk (provided by FamilyMart Co., Korea) to prepare a composite colloidal solution. When 125 ml of a gas-containing aqueous solution (Kin Cider) fell freely down the composite colloidal solution, the following measurement results were obtained: Vmax=340 ml, Hmax=12 cm, R0.5 (H)=10.8:3.6:7.2, R1 (H)=9.6:4.8:4.8, R5 (H)=9:6:3, R10 (H)=7.2:6:1.2. About 30 minutes after the free fall, a solid structure in the form of a foam crust was obtained.

### Example 7

5 g of fiporog B-10 was homogeneously mixed with 5 g of sugar to obtain a powder. The powder was added to 30 ml of E+ Supgol Milk (provided by FamilyMart Co., Korea) to prepare a composite colloidal solution. When 125 ml of a gas-containing aqueous solution (Kin Cider) fell freely down the composite colloidal solution, the following measurement results were obtained: Vmax=340 ml, Hmax=12 cm, R0.5 (V)=12:4:8, R1 (V)=11.5:4.5:7, R5 (V)=10.8:5.4:5.4, R10 (V)=9.6:6:3.6. About 30 minutes after the free fall, a solid structure in the form of a foam crust was obtained.

### Example 8

2.5 g of fiporog B-10 was homogeneously mixed with 2.5 g of sugar to obtain a powder. The powder was added to 20 ml of E+ Supgol Milk (provided by FamilyMart Co., Korea) to prepare a composite colloidal solution. When 125 ml of a gas-containing aqueous solution (Coca-Cola Zero) fell freely down the composite colloidal solution, the following measurement results were obtained: Vmax=340 ml, Hmax=12 cm, R0.5 (H)=12:3:9, R1 (H)=11.4:3.8:7.6, R5 (H)=9.6:3.8:5.8, R10 (H)=8.4:4.2:4.2. About 30 minutes after the free fall, a solid structure in the form of a foam crust was obtained.

### Example 9

2.5 g of fiporog B-10 was homogeneously mixed with 2.5 g of sugar to obtain a powder. The powder was added to 15 ml of E+ Supgol Milk (provided by FamilyMart Co., Korea) to prepare a composite colloidal solution. When 125 ml of a gas-containing aqueous solution (Kin Cider) fell freely down the composite colloidal solution, the following measurement results were obtained: Vmax=340 ml, Hmax=12 cm, R0.5 (V)=12:3.4:8.6, R1 (V)=10.8:3.8:7, R5 (V)=9.6:3.8:5.8, R10 (V)=8.4:4.4:4.0. About 30 minutes after the free fall, a solid structure in the form of a foam crust was obtained.

### Example 10

10 g of fiporog A-100 was homogeneously mixed with 20 g of sugar to obtain a powder. The powder was added to 50 ml of Pasteur Organic Milk (produced by Pasteur Milk Co., Korea) to prepare a composite colloidal solution. When 100 ml of a gas-containing aqueous solution (natural soda pop) fell freely down the composite colloidal solution, the following measurement results were obtained: Vmax=340 ml, Hmax=12 cm, R0.5 (H)=11:4.5:6.5, R1 (H)=10:5.3:4.7, R5 (H)=7.5:6:1.5, R10 (H)=6.8:6.1:0.7. About 30 minutes after the free fall, a solid structure in the form of a foam crust was obtained.

### Example 11

10 g of fiporog A-10, 20 g of sugar and 1 g of a coffee concentration powder were homogeneously mixed together to obtain a powder. The powder was added to 50 ml of Pasteur Organic Milk (produced by Pasteur Milk Co., Korea) to prepare a composite colloidal solution. When 100 ml of a gas-containing aqueous solution (natural soda pop) fell freely down the composite colloidal solution, the following measurement results were obtained: Vmax=340 ml, Hmax=12 cm, R0.5 (V)=12:3.8:8.2, R1 (V)=11.4:5:6.4, R5 (V)=8.6:5.6:3.0, R10 (V)=7.7:5.7:2.0. About 30 minutes after the free fall, a solid structure in the form of a foam crust was obtained.

### Example 12

10 g of fiporog A-10 was homogeneously mixed with 20 g of yellowish white sugar to obtain a powder. The powder was added to 50 ml of Pasteur Organic Milk (produced by Pasteur Milk Co., Korea) to prepare a composite colloidal solution. When 150 ml of a gas-containing aqueous solution (Fanta) fell freely down the composite colloidal solution, the following measurement results were obtained: Vmax=340 ml, Hmax=12 cm, R0.5 (H)=12:1.2:10.8, R1 (H)=12:2.7:9.3, R5 (H)=12:4.8:7.2, R10 (H)=12:5.8:6.2. About 30 minutes after the free fall, a solid structure having a height of about 6 cm in the form of a foam crust was obtained. About 30 minutes after the free fall, the degree of clearness of the solution state was evaluated to be ‘poor’. About 3 hours after the free fall, the degree of clearness of the solution state was evaluated to be ‘average’. About 12 hours after the free fall, the degree of clearness of the solution state was evaluated to be ‘good’.

### Example 13

10 g of fiporog C-10, 20 g of sugar and 1 g of a coffee concentration powder were homogeneously mixed together to obtain a powder. The powder was added to 50 ml of Pasteur Fresh Milk (produced by Pasteur Milk Co., Korea) to prepare a composite colloidal solution. When 100 ml of an overcooled gas-containing aqueous solution (Mineral water produced from Chojeong-ri, Chungcheongbuk-do, Korea) fell freely down the composite colloidal solution, the following measurement results were obtained: Vmax=340 ml, Hmax=12 cm, R0.5 (V)=12:3.4:8.6, R1 (V)=10.8:3.8:7, R5 (V)=9.6:3.8:5.8, R10 (V)=8.4:4.4:4.0. A solid structure in the form of an icy foam crust was obtained.

### Example 14

10 g of fiporog C-100, 20 g of sugar, 1 g of a coffee concentration powder and 30 g of dry ice were homogeneously mixed together to obtain a powder. The powder was added to 50 ml of Pasteur Fresh Milk (produced by Pasteur Milk Co., Korea) to prepare a composite colloidal solution. When 50 ml of a gas-containing aqueous solution (Mineral water produced from Chojeong-ri, Chungcheongbuk-do, Korea) fell freely down the composite colloidal solution, the following measurement results were obtained: Vmax=340 ml, Hmax=12 cm, R0.5 (H)=12:3:9, R1 (H)=11.4:3.8:7.6, R5 (H)=9.6:3.8:5.8, R10 (H)=8.4:4.2:4.2. A solid structure in the form of an icy foam crust was obtained.

As apparent from the above description, the present invention provides a new concept of bubble drink (foamy drink). The bubble drink of the present invention is produced by primarily processing a food in the form of a powder catalyst promoting foaming, secondarily processing the processed food to obtain a concentrate in the form of a composite colloidal solution, and reacting the concentrate with a gas-containing saturated aqueous solution to convert all reactants into a foam structure. The bubble drink is ingested together with the gas contained in the foam when drinking. This is a concept of ‘bubble drink’.

The final product produced by a bubbling engineering process in the present invention may be a structure consisting of three states, a foam scum, a brewed solution (patterned water), a foam-stabilized structure, or a combination thereof. That is, the ‘bubble drink’ of the present invention is an instant food produced by converting materials to be ingested present in three composite states, i.e. solid, liquid and gas states, converted into a foam structure.

Since the ingredients of the catalytic material and the concentrate, which are used in the final foam-generating step, are selected and their contents are controlled, the characteristics of the bubble drink can be adjusted. Accordingly, the bubble drink of the present invention can be provided as a custom-made or custom-ordered product that exhibits various characteristics. Therefore, an ingestion method using the bubble drink of the present invention is highly industrially applicable and effective.

The bubble drink of the present invention offers the following expectable industrial applications.

i) Since the bubble drink of the present invention is a perfect food, it creates diet food industries for infants, old people, patients and athletes.

ii) Since the bubble drink of the present invention is a new concept of drink that stimulates curiosity, it is expected to open new drink markets. Particularly, since the colloidal solution as a food additive catalyzing the generation of air bubbles in the bubble drink can be preserved for a long time and has a volume one-tenth the volume of the gas-containing aqueous solution, the bubble drink can be processed into an extract, packaged in a tube, a can or a glass bottle, and exported. Furthermore, milk and a grain powder constituting the colloidal solution are ingredients that can be readily fermented. Accordingly, alcoholic fermentation or lactic acid bacteria fermentation can be performed on the colloidal solution to produce new fermented drinks that are ingested in the form of bubble drinks.

iii) The bubble drink of the present invention can open a new market in the application of healthy foods. Since air makes up 50% or more of the volume of the bubble drink according to the present invention, even a very small amount of the bubble drink can offer a sense of satiety and can greatly relieve thirst. Moreover, the bubble drink of the present invention can be used to develop a health auxiliary of diet food that prevents and treating obesity and diabetes.

Having described the present invention, it will be apparent that many changes and modifications may be made to the above-described embodiments without departing from the spirit and the scope of the present invention.

What is claimed is:

1) A bubble drink as a health food, which is produced by imparting a catalytic function for gas escaping to an adsorptive fine food powder to allow the fine food powder to be dispersed in a colloidal solution reverse vessel, and reacting the dispersion with a gas-saturated solution to generate foam, whereby the food is converted into foam having a three-state composite structure of gas, liquid and solid states suitable to be ingested within a digestive system.

2) The bubble drink according to claim 1, wherein a saccharine crystalline (crystalloid) powder having a size of 10 μm or less is further added to the fine food powder.

3) The bubble drink according to claim 1, wherein the colloidal solution is prepared by dispersing the fine food powder in a composite colloidal solution of emulsion-suspension to obtain a food concentrate in a composite colloidal state and instantaneously reacting the food concentrate with a solution saturated with CO₂ to convert the entire solution into foam having a diameter of 2 mm or less.

4) The bubble drink according to claim 1, wherein the gas-saturated solution contains CO₂ in the state of a gas, a dry ice powder, a liquid or a composite thereof.

5) The bubble drink according to claim 2, wherein a functional material is added depending on the drinking purposes, functions and tastes during mixing of the fine food powder and the saccharine crystalloid powder.

6) The bubble drink according to claim 5, wherein a dry ice powder is added during mixing.

7) A method for producing a bubble drink that is provided as a custom-made or custom-ordered product to maintain or improve the health of lives and is ingested for various purposes, the method comprising the steps of:
a) steaming and drying or slightly parching natural grains to obtain a parched cereal powder, pulverizing the parched cereal powder into a fine powder of roasted grains for bubble drink while maintaining the humidity at a level lower than 5%, and dividing the fine powder of roasted grains into powders on the basis of size (>100 μm, 100 μm-10 μm, 10 μm-1 μm, and 1 μm-1 nm) as a first step;
b) pulverizing a crystalline powder or granular crystal of a monosaccharide or oligosaccharide selected from solid substances including sugar, fructose, glucose, lactose, starch sugar, oligosaccharide, dextrin, -starch, D-mannitol and xylitol to prepare a saccharine crystalloid powder having a size of 10 μm or less as a second step;
c) adsorbing and distributing the powders prepared in the first and second steps in a wind tunnel to obtain a powder having a particle size of 10 μm or less, adding a functional material to the powder, and mixing the mixture with a colloidal solution in the state of a protein emulsion, such as milk, to prepare a food concentrate in a gel state as a third step; and,
pulverizing, rotating or swirling the food concentrate to convert the gel into a sol, and freely dropping a gas-saturated solution on the sol to generate foam as a fourth step.

8) The method according to claim 7, wherein, during the preparation of the crystalloid powder (second step), the structure of the colloidal dispersion medium is designed in a composite manner such that the respective mixing reactions, such as replacement, diffusion and dispersion, are controlled according to the purpose of additives; the saccharine crystalloid powder is divided into powders on the basis of size (100 μm-1 nm) and hydrated; and the hydrate is converted into a gel state in a colloid reserve vessel in the state of emulsion-suspension and diluted to prepare a sol, so that the structure of the gel colloid is aggregated and aged with the passage of time.

9) The method according to claim 7, wherein, in the third step, the functional material is processed into a state of a fine powder or colloidal solution suitable for a bubbling engineering process before addition.

10) The method according to claim 7, wherein, in the third step, the gelling is carried out by dripping and rolling drops of the colloidal solution on an inclined surface of the powder to infiltrate the colloidal solution into the powder.

11) The method according to claim 7, wherein, in the course of adsorption using the wind tunnel, high-pressure low-temperature liquid carbon dioxide is sprayed to improve the performance of the wind tunnel so that the temperature, pressure, humidity, air stream distribution, particle size, and constituent ratio and binding state of the powders are controlled, and at the same time, powder processing in a frozen state under ambient pressure is carried out to form a fine powder mixture with dry ice, thereby improving the preservation of the food and ensuring storage and transport stability.

12) The method according to claim 7, wherein, in the fourth step, the bubbling engineering process is carried out through the sub-steps of processing the raw materials into a powder having a particular particle size, dispersing the powder in the colloidal solution, and reacting a gas-saturated solution with the colloidal dispersion.

13) The method according to claim 7, wherein, in the fourth step, free fall and vortex turbulence are employed as aeration triggers for colloidal explosive reactions so that the height and amount of the free fall are controlled to adjust an increase in the entropy of the bubble colloid, and at the same time, a gravity-free state is imparted to the gas-saturated drink to achieve uniform and fine foaming during the free fall.

14) A food concentrate of a bubble drink, comprising:
a fine powder of roasted grains having a humidity lower than 5% prepared by steaming and drying or slightly parching natural grains, and pulverizing the dried or parched natural grains;
a saccharine crystalloid powder having a size of 10 μm or less prepared by pulverizing a crystalline powder or granular crystal of a monosaccharide or oligosaccharide selected from sugar, fructose, lactose, starch sugar, oligosaccharide, dextrin, α-starch and D-mannitol; and,
a food concentrate in a gel state prepared by mixing an additive with a colloidal solution in the state of a protein emulsion, such as milk, to impart desired functions to the bubble drink and gelling the mixture.

15) A bubble drink for dietary treatment of a disease, such as obesity or diabetes, that is ingested so as to control the caloric intake of a consumer while offering a sense of satiety to the consumer wherein the bubble drink is produced by a method comprising the steps of:
steaming and drying or slightly parching natural grains to obtain a parched cereal powder, pulverizing the parched cereal powder into a fine powder of roasted grains for bubble drink while maintaining the humidity at a level lower than 5%, and adjusting the amount of the fine powder of roasted grains to the caloric intake of a consumer (first step);
pulverizing a crystalline powder or granular crystal of a monosaccharide or oligosaccharide selected from solid substances including sugar, fructose, glucose, lactose, starch sugar, oligosaccharide, dextrin, -starch, D-mannitol and xylitol to prepare a saccharine crystalloid powder having a size of 10 μm or less (second step);
adding a functional material while adsorbing and distributing the powders prepared in the first and second steps in a wind tunnel to obtain fine powders for colloid on the basis of size (>100 μm, 100 μm-10 μm, 10 μm-1 μm, and μm-1 mm), and mixing the mixture with a colloidal solution in the state of a protein emulsion, such as milk, to prepare a food concentrate for diet in a gel state (third step); and,
pulverizing, rotating or swirling the food concentrate to convert the gel into a sol, and freely dropping a gas-saturated solution on the sol to generate foam (a bubbling engineering process) (fourth step).

16) A method for ingesting a pharmacologically active ingredient by a bubbling engineering process to improve the health of lives, treat a disease or provide pharmacological effects, the method comprising the steps of:
a) pulverizing a pharmacologically active drug prescribed and provided by a doctor and a pharmacist to prepare a fine powder having a size of 10 μm or less (first step);
b) pulverizing a crystalline powder or granular crystal of a monosaccharide or oligosaccharide selected from solid substances including sugar, fructose, glucose, lactose, starch sugar, oligosaccharide, dextrin, -starch, D-mannitol and xylitol to prepare a saccharine crystalloid powder having a size of 10 μm or less (second step);
c) adsorbing the fine drug powder prepared in the first step to the saccharine crystalloid powder prepared in the second step to obtain a powder having a particle size of 10 μm or less, adding a functional material to the powder, and,
d) mixing the mixture with a colloidal solution in the state of a protein emulsion, such as milk, to prepare a drug concentrate in a gel state (third step); and pulverizing, rotating or swirling the drug concentrate to convert the gel into a sol, and freely dropping a gas-saturated solution on the sol to generate foam (a bubbling engineering process) (fourth step).
